(19) United States
(12) Patent Application Publication
(10) Pub. No.: US 2007/0132596 A1
(43) Pub. Date: Jun. 14, 2007

(54) Sound Privacy Machine and Methods of Use Thereof

(75) Inventor: Lewis Green, Aventura, FL (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: LG Home Products, LLC, Aventura, FL (US)

(21) Appl. No.: 11/539,032

(22) Filed: Oct. 5, 2006

Related U.S. Application Data
(60) Provisional application No. 60/724,001, filed on Oct. 5, 2005.

(51) Int. Cl. G08B 23/00; A61F 11/06

(52) U.S. Cl. 340/573.1; 381/71.1

(57) ABSTRACT

The present invention relates to masking airborne sound transmissions and is particularly directed toward masking bodily function sounds emanating from a bathroom. To that end the present invention provides a sound system comprising a sound generator in wireless communication with a transmitter that detects motion. In automatic function, the transmitter is affixed to or in close proximity to a toilet so when the toilet is in use a signal is transmitted to the sound generator which then produces masking sounds at a predetermined volume.

### CROSS-REFERENCE TO RELATED APPLICATION

This application claims the benefit of priority of co-pending U.S. Provisional Patent Application Ser. No. 60/724,001 entitled “A Sound Privacy Machine and Methods of Use Thereof” filed Oct. 5, 2005. Priority of the filing date of Oct. 5, 2005 is hereby claimed, and the disclosure of the Provisional Patent Application is hereby incorporated by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

1. Field of the Invention

The present invention relates to masking airborne sound transmissions and is particularly directed toward masking bodily function sounds emanating from a bathroom.

2. Description of the Related Art

A disturbing and ever-present problem in contemporary living is the increasing proliferation of unwanted sound or noise. Sound striking a wall or ceiling surface is transmitted through the air in the wall or ceiling cavity. It then strikes the opposite wall surface, causing it to vibrate and transmit the sound into the adjoining room. Sound also is transmitted through any openings going into the room, such as air ducts, electrical outlets, window openings, and doors. This is airborne sound transmission.

Now consider walking into a room and feeling anxious, despite your most logical efforts to tell yourself to relax. Imagine feeling your whole body becoming tense and your breathing becoming rapid. Imagine the room you are in is a bathroom in your own home or a friend's home and that you are anxious about family members or friends hearing your bodily function sounds emanating from the bathroom. This anxiety affects millions of people and ranges in severity from simple embarrassment to a condition known as paruresis.

Paruresis, also known as pee shy, shy kidney, or shy bladder syndrome, is a type of social anxiety disorder in which the sufferer is unable to urinate in the (real or imaginary) presence of others. It goes beyond simple embarrassment or a desire for privacy in that it is much more severe and may cause unnecessary inconvenience, because the inability to urinate, although psychological in origin, is physical in its effect, and not under the control of the sufferer.

Many factors inhibit a paruretic's ability to use a bathroom, public or private, one of which is sound transmission. A paruretic is keenly sensitive to the sounds of elimination, both his or her own sounds, as well as the sounds of others. Even if the configuration of toilets in a public restroom is sufficient for a paruretic to feel sufficiently reassured to use the facility, the fear of producing sounds or hearing others' sounds may prevent the paruretic from being successful in the public bathroom. The paruretic is often no better off in a private residence with the bathroom door closed, because the fear of producing sounds may prevent the paruretic from being successful in the bathroom.

A national co-morbidity study conducted in the United States indicates that approximately 7% of the U.S. population suffers from this condition, or roughly 17 million Americans. Of those, 1 to 2 million suffer from paruresis in its severe form, significantly interfering with their work, social relationships, travel, and other important life activities. While there are no international studies currently available on the subject, anecdotal evidence seems to suggest that the 7% figure is fairly representative across populations in many diverse cultures worldwide. It is hard to collect population figures on this subject because people who have it rarely disclose it. Lack of funding has similarly had a negative impact on the ability to conduct studies and acquire information.

It is well documented that many Japanese women are embarrassed at the thought that someone else can hear them while they are using the toilet. To cover the sound of bodily functions, many women flushed public toilets continuously while using them, wasting a large amount of water in the process. As education campaigns did not stop this practice, a device was introduced in the 1980s that, after activation, produces the sound of flushing water without the need for actual flushing. One brand name commonly found is the Otohime, which literally means Sound Princess. This device is now routinely placed in most new public women's rooms in Japan, and many older public women's rooms have been upgraded. The Otohime may be either a separate battery-operated device attached to the wall of the toilet, or included in an existing washlet. The device is activated by pressing a button, or by the wave of a hand in front of a motion sensor. After activation, the device creates a loud flushing sound similar to a toilet being flushed. This sound either stops after a preset time or can be halted through a second press on the button. It is estimated that this saves up to 20 liters of water per use. However, some women believe that the Otohime sounds artificial and prefer to use a continuous flushing of the toilet instead of the recorded flush of the Otohime. Although devices such as the Otohime can be incorporated into new construction for public facilities, such devices are often difficult to retrofit to existing restrooms. Such devices are less frequently incorporated into residential bathrooms, due to cost and installation requirements.

Whether one suffers from paruresis or is simply embarrassed by the knowledge that others can hear their bodily function sounds emanating from the bathroom, it is clear that people suffering from this type of social anxiety need help. The use of sound devices that can mask such sounds can be helpful. Devices that are simple to use, adaptable for use in the home, and dependable are key to solving this world-wide problem.

### SUMMARY

In accordance with the invention, an apparatus provides masking sounds in the vicinity of a toilet and includes a detector positioned in the vicinity of a toilet, wherein said detector comprises a transmitter, and a receiver that is in direct wireless communication with said detector and thereby activates a sound system, wherein said sound system is capable of emitting music or natural sounds. The detector can be positioned on the lid of a toilet such that, when the toilet lid is in a horizontal position, the detector communicates to the receiver a signal that turns the sound system off, and when the lid is in a vertical position, the detector communicates a signal to the receiver that turns the sound system on. In this way, the apparatus can mask unwanted sounds, such as bodily function sounds emanating from a bathroom.

In one aspect, the apparatus provides a sound system comprising a sound generator in wireless communication with a transmitter that detects position. The transmitter is affixed to, or in close proximity to, a toilet lid or seat so that, when the toilet is in use, a signal is automatically transmitted to the sound generator, which then produces masking sounds at a predetermined volume. When the toilet is not in use, the sound generator does not produce the masking sounds.

In another aspect, the apparatus can be configured to control sound activation and deactivation so as to encourage desired action involving the toilet. For example, the transmitter can be positioned on or near the toilet lid or seat and the sound generator can be activated (switched “on”) only when the seat is raised and deactivated (switched “off”) only when the seat is lowered. Once the lid or seat is in the raised position and the sound generator is activated, the sound generator will continue to produce sounds until the lid or seat is lowered. In this way, persons are reminded to lower the toilet lid after use.

Additional advantages and novel features of this invention shall be set forth in part in the description that follows, and in part will become apparent to those skilled in the art upon examination of the following specification or may be learned by the practice of the invention. The advantages of the invention may be realized and attained by means of the instrumentalities, combinations, compositions, and methods particularly pointed out in the appended claims.

### BRIEF DESCRIPTION OF THE DRAWINGS

The accompanying drawings, which are incorporated herein and form a part of the specification, illustrate non-limiting embodiments of the present invention, and together with the description, serve to explain the principles of the invention.

FIG. 1 illustrates a perspective view of a system constructed in accordance with the present invention.

FIG. 2 illustrates the detector of the FIG. 1 system attached to a toilet lid.

FIG. 3 illustrates movement of the toilet lid to a vertical or activation position for the FIG. 1 system.

FIG. 4 is a block diagram of the FIG. 1 system.

FIG. 5 is a schematic representation of the circuitry for the detector of the FIG. 1 system.

FIG. 6 is a schematic representation of the circuitry for the sound system of the FIG. 1 system.

FIG. 7 is a block diagram of the control module for another embodiment of a system constructed in accordance with the present invention.

FIG. 8 illustrates a toilet lid with a control module and sensor module constructed in accordance with the invention.

FIG. 9 illustrates the underside of the FIG. 8 toilet lid.

FIG. 10 illustrates a toilet lid with a control module and pressure sensor and motion sensor constructed in accordance with the present invention.

FIG. 11 illustrates a toilet lid and seat combination with a control module and pressure sensor and motion sensor constructed in accordance with the present invention.

### DETAILED DESCRIPTION

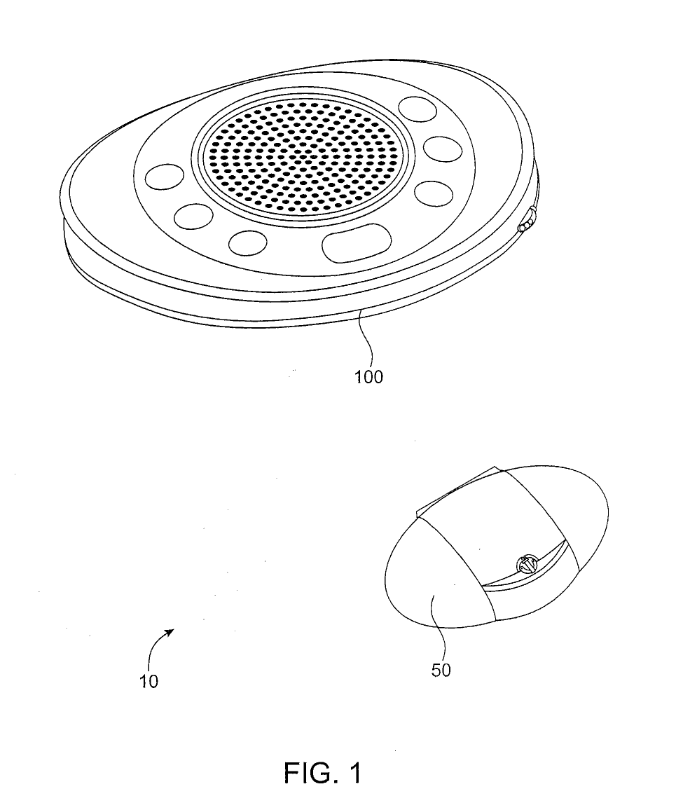

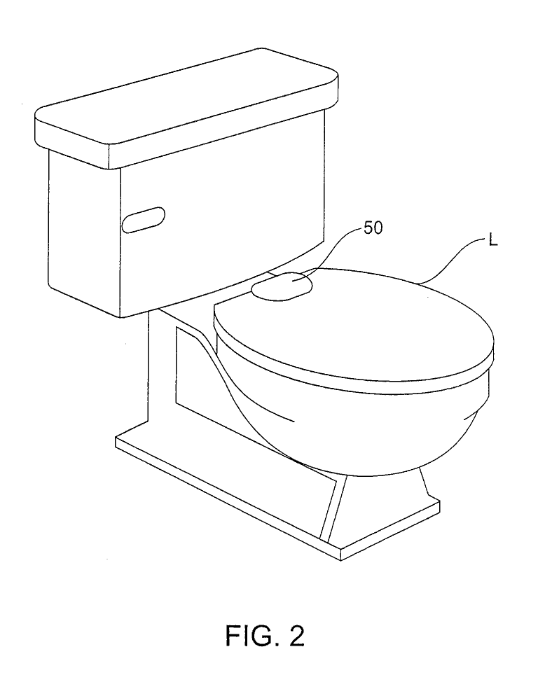

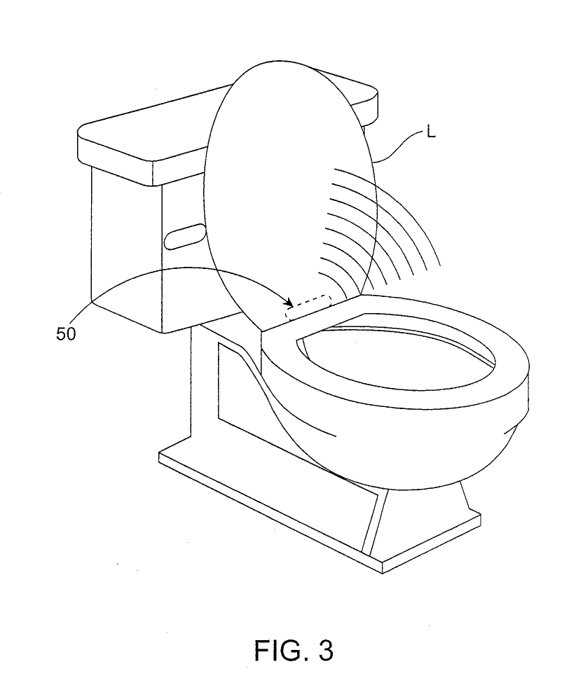

A sound privacy machine 10 according to this invention is shown in FIG. 1. The sound privacy machine 10 comprises a detector 50 and an electronic sound system 100 in operative communication with one another. The detector 50 is attached to a toilet, preferably on either the toilet lid or the toilet seat, however attachment to other places on the toilet or in the vicinity of the toilet are contemplated. As shown in FIG. 2, the detector 50 is attached to the toilet lid L by means of an adhesive; however, attachment can result by a number of means, such as but not limited to, screws, clips, VELCRO® or other convenient fastening mechanisms. When a person approaches the toilet for use, the person raises the toilet lid L, as shown in FIG. 3, and the detector 50 detects the change in position and generates a signal that is transmitted to the sound system 100. The sound system 100 receives the signal, is activated or switched on, and sound is emitted at a predetermined volume, thus masking the sound of eliminating, such as urinating or bowel movements. The emitted sound may be in the form of, but is not limited to, music, rain, surf, or other suitable masking sound. The volume of the sound can be set to a desired loudness. Upon finishing the use of the toilet, the user lowers the lid L and the detector 50 detects the change in position and generates another signal. The sound system 100 receives the second signal, is deactivated or switched off, and ceases to emit the sound.

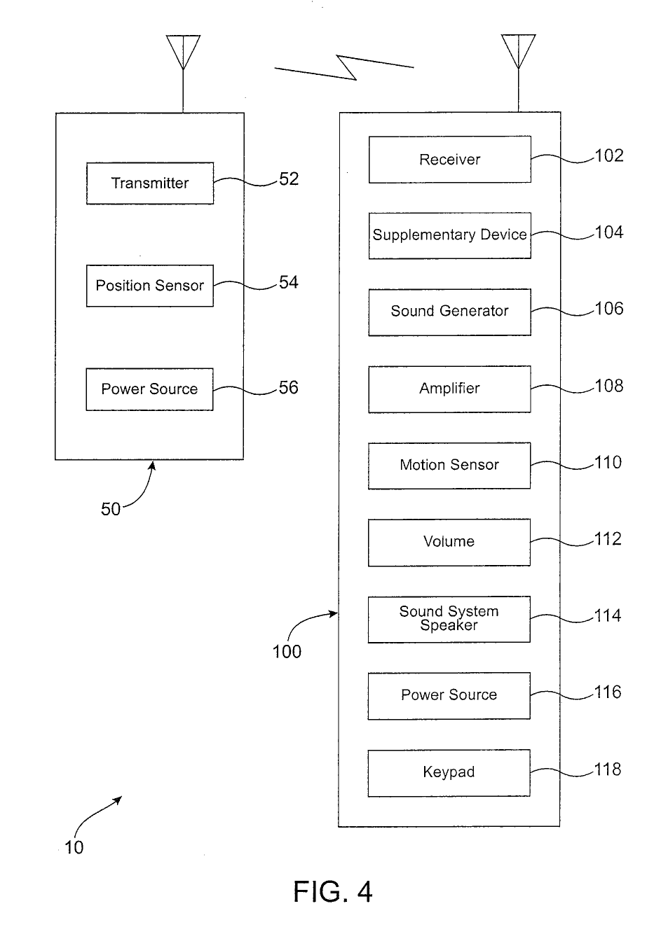

The detector 50 as shown in FIG. 4 comprises a transmitter 52, a position sensor 54, and a power source 56. The transmitter 52 sends a signal that directly or indirectly activates (switches “on”) and deactivates (switches “off”) the sound system 100, in response to the position sensor 54 sensing the occurrence of a change of environment. A battery may be used as the power source in view of greater flexibility in placement of the device without requiring changes to electrical wiring and in view of safety concerns in places where water is used.

In one embodiment, the detector 50 incorporates a tilt switch that serves as a position detector. Although a wide variety of tilt switches are known in the art, only a few will be discussed here and this is not intended to limit the scope of those switches that may be useful in the detector 50. Some tilt switches are intended to detect very slight changes in the attitude of the switch and respond to small changes in the angle of tilt. For example, perhaps the most well known switch, is the mercury tilt switch which utilizes a globule of mercury in a sealed vial which when tilted, causes the mercury to move from one end of the vial to the other and to conductively bridge two conductors which are disposed within the vial. The mercury switch can detect changes in attitude as small as one or two degrees. In some applications, however, the mercury switch cannot be used, due to health concerns over the use of mercury. In addition, the mercury switch can be more sensitive than is desired, causing a decrease in ease of use and in dependable switch action. For use in toilet applications, the detector 50 detects change in position, producing an output in response to a change in orientation or location of the detector, regardless of the force or velocity of position change.

Whether a user raises or lowers the toilet lid swiftly or slowly, gently or harshly, the detector 50 will detect the change in position of the toilet lid from closed (horizontal) to open (vertical) and will generate its output. This feature is effectively achieved with a tilt switch for the detector 50 that comprises a ball switch, in which a conductive ball is enclosed within a housing having opposite electrical contacts that are completed upon the conductive ball rolling, by gravity, into position bridging the electrical contacts. Such switches are typically referred to as a ball bearing tilt switch. A switch of this type responds to changes in attitude or inclination between horizontal and vertical, and works well in the toilet application of the present invention, since the toilet lid L and/or seat S are moving from a closed or lowered, near-horizontal position to an open or raised, near-vertical position (that is, through a change in inclination of about 90 degrees). In one configuration, two pairs of parallel ball tilt switches are provided, one pair configured to detect (be switched on) when in the horizontal position, and another pair configured to detect (be switched on) when in the vertical position. Using a pair of tilt switches ensures dependable switch action that is independent of force and velocity of moving the toilet lid and/or seat. When properly connected, the ball tilt switches work in conjunction to ensure appropriate signal output for the detector 50. Those skilled in the art will understand the proper construction and configuration of switches in view of the description herein.

Thus, the detector 50 comprises a tilt switch that activates and deactivates the sound system 100 in response to the inclination of the tilt switch. In the FIG. 2 embodiment, the detector 50 is mounted to a toilet lid L or toilet seat S and only the raising and lowering of the lid L or seat S (shown in FIG. 3) will generate a transmitted output signal from the detector 50. That is, when the lid L and seat S are in the lowered position, no sound will emanate from the sound system. When the lid L (if the lid has a detector) and the seat S (if the seat has a detector) are moved to the raised, vertical position, the detector generates its output signal and the sound system 100 is activated or turned on, and a predetermined masking sound will emanate from the system at a predetermined volume. The sound will continue to be heard until the lid L (and/or seat S, depending on detector configuration) is lowered. In this way, the illustrated configuration not only aids with masking the sounds of eliminating, but can also serve as a reminder for the user to return the lid L and/or seat S from the raised (vertical) position to the original lowered (horizontal) position after use, because sounds will continue to emanate from the sound system 100 until the lid L and/or seat S are returned to the lowered position.

The sound system 100 may comprise a sound generator 106 for playing a variety of stored (prerecorded) masking sounds, such as music or nature sounds. Alternatively, the sound system 100 may further comprise a supplementary device 104 such as a CD player/radio turner or MP3 player, for playing a greater variety of sounds that can mask the noises of elimination. The supplementary device may incorporate the ability to dispense a fragrance, or may comprise an ion or electrostatic air cleaner activated for a predetermined time, or the like. In addition, the sound system 100 further comprises an amplifier 108, motion sensor 110, volume control 113, speaker 114, and power source 116. The sound system 100 may operate independently from the detector 50 as a stand-alone unit that an operator could place on the bathroom sink and manually turn on and off. The sound system 100 also can include a keypad 118 that can be activated by the user to set options and user preferences. The sound system 100 is located sufficiently close to the toilet and detector 50 so the sound system and detector can be in wireless communication and so the generated sounds serve as masking sounds.

In an alternative embodiment, the detector 50 comprises a position sensor 110 that radiates light, such as, but not limited to, infrared or laser light. As such, the detector 50 may be situated in the vicinity of the toilet in a fixed position. Infrared rays are radiated toward the toilet, the presence of a person in the vicinity of the toilet is detected based on how the infrared rays are reflected. When the person has been detected, the transmitter 52 communicates a signal to the receiver 102 and the sound system 100 is activated. In this way, the detector detects the position of a person relative to the toilet and in generates a suitable output signal. Sensors that operate in this fashion are well-known in the art. Essentially, a microcomputer uses the output from a drive circuit to radiate infrared rays from an infrared light transmitter toward the body of a user. The reflected light is detected by a light receiver, amplified by an amplifier and the microcomputer reads the result of a comparison performed by a comparator. Then a transmitter generates a signal that is communicated to a receiver and the sound system 100 is activated. When no motion is detected another signal is generated and the sound system 100 is turned off.

In another embodiment the detector 50 may comprise a sensor that detects pressure. In this embodiment the detector 50 may be shaped like a pad and placed on the floor in front of the toilet and when the user steps on the pad a signal will be generated and transmitted to the sound system 100. Alternatively, the pressure switch may be placed under the toilet seat in contact with the surface of the toilet rim. In this configuration, when the user sits on the toilet seat a signal will be generated and transmitted to the sound system 100. Finally, the detector 50 may be attached to the toilet tank, so that when the lid L is “opened” or raised to a vertical position, it rests against the toilet tank, such that pressure is subjected to the detector 50 and a signal is generated and transmitted to the sound system 100. Again, the activated state of the detector will remain until the raised lid is returned to the lowered position.

In another alternate embodiment the present invention contemplates the use of a detector 50 and a switchable power receptacle. In this embodiment any standard electronic device such as but not limited to a radio, CD/MP3 player, or sound generator may be plugged into the switchable power receptacle. The power receptacle in this instance comprises a receiver and when a signal is received from the detector 50 (described previously) power is supplied to the electronic device and a masking sound is generated. When the user leaves the bathroom or closes the toilet seat, the detector 50 transmits another signal and the switchable power supply ceases the supply of power, in effect turning off the electronic device.

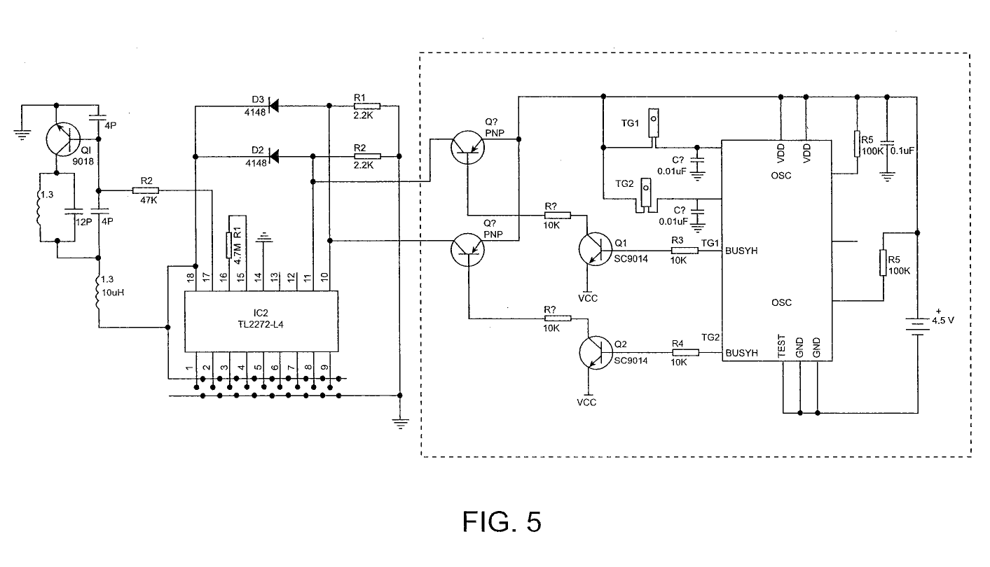

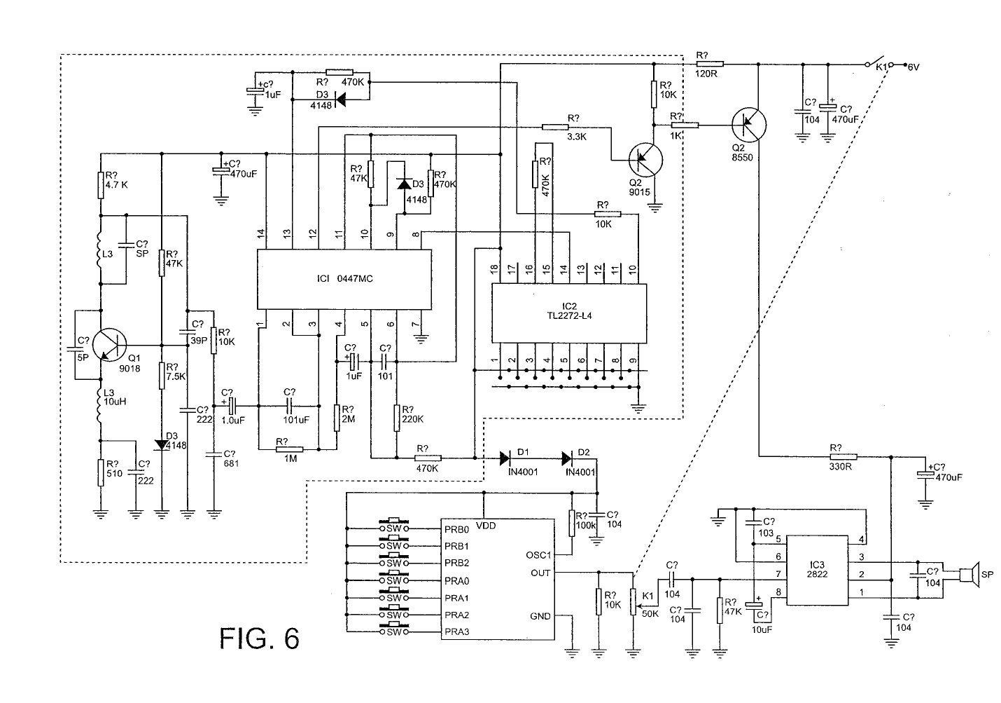

FIG. 5 is a schematic representation of the circuitry for the detector of the FIG. 1 system. FIG. 6 is a schematic representation of the circuitry for the sound system of the FIG. 1 system. Those skilled in the art will understand that many different configurations of circuitry are possible for implementing the components and operation described herein. Those skilled in the art will be able to construct such alternate configurations in view of the description herein.

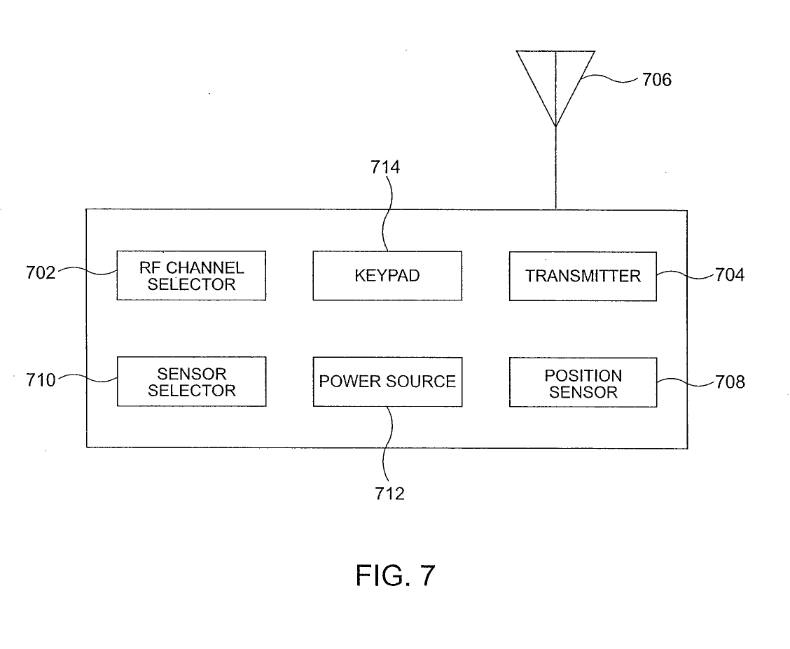

FIG. 7 is a block diagram of a detector 700 for another embodiment of a system constructed in accordance with the present invention. The FIG. 7 detector 700 controls operation of an associated sound module, such as the sound module 100 having the construction illustrated in FIG. 4. The detector 700 includes an RF channel selector 702, a transmitter 704, and an antenna 706 for communications with the associated sound module via wireless communication. Alternatively, the detector may communicate with the sound module by infrared or laser means, as described above.

The detector module 700 also includes a motion sensor 708, a sensor selector 710, and a power source 712. The power source supplies energy to the various components that require it for operation. The motion sensor may comprise one or more transducers that can detect motion of a bathroom fixture, such as the toilet lid, seat, or door, and can detect motion in the vicinity of the bathroom fixture, such as the presence of a person moving about. Thus, depending on the configuration of the system comprising sound module and detector module 700, movement of the toilet lid and/or seat itself can be detected, and motion of the bathroom door or stall door can be detected, and movement of a person in the area of the toilet can be detected. The system can be configured to detect all types of such motion or presence, or can be configured to detect any one or more of the types of motion or presence. The detector module 700 also can include a keypad 714 that can be activated by the user to set options and user preferences.

The sensor selector 710 determines which one of the various motion and presence sensors have been triggered or activated, if more than one such sensor is provided, and determines the appropriate response. For example, the sensor selector can be configured to give priority to one selector over others, or can be configured to follow operational logic such as activating the sound module only if a particular sequence of detection is observed. In this way, for example, the sensor selector of the control module can be configured to activate the sound module if door motion is followed by lid motion and/or seat motion and/or presence of a person, and to not activate the sound module if lid motion is followed by door motion and/or the absence of a person.

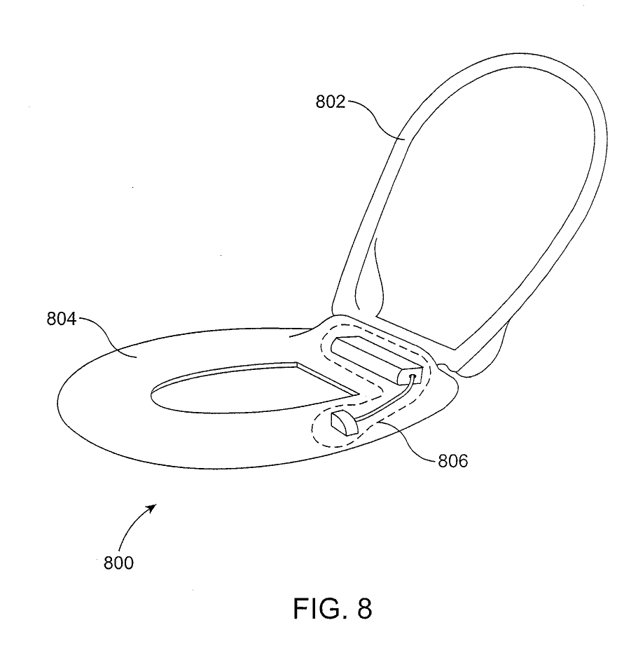

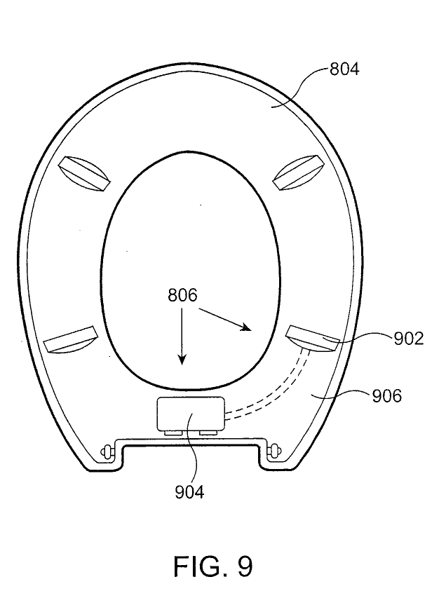

FIG. 8 illustrates a toilet seat/lid combination 800 for a sound privacy system constructed in accordance with the present invention. In the system 800 illustrated in FIG. 8, a toilet lid 802 is hinged to the toilet seat 804 and a detector 806 (indicated by the dashed line) is affixed to the toilet seat. Alternatively, the detector 806 may be built into or embedded in the toilet seat. FIG. 9 illustrates the underside of the FIG. 8 toilet seat 804. FIG. 9 shows that the detector 806 includes a pressure sensor 902 and a control module 904 connected by a wire 906, which is embedded within the toilet seat (as indicated by the dashed line in FIG. 9). The connecting wire 906 permits signals to be communicated between the pressure sensor and control module. Alternatively, the connecting wire 906 could be mounted externally to the toilet seat, or signals can be communicated by means of a wireless connection between the pressure sensor and control module. The control module 904 can include an access panel that permits adjustment of operational settings and routine maintenance such as replacing a battery and the like.

In the system of FIG. 8 and FIG. 9, as with the previously described embodiments, the detector 806 is in direct wireless communication with the sound system 100 (FIG. 4) for controlling activation of predetermined masking sounds, such as music. When the sensor 902 is triggered or activated in accordance with the configured control module operation, the masking sounds are generated from the sound module. It should be understood that the detector 806 controls operation of an associated sound module, such as the sound module 100 having the construction illustrated in FIG. 4. The detector 806 includes an RF channel selector, a transmitter, and an antenna as illustrated above for the FIG. 7 block diagram, so the detector can communicate with the associated sound module via wireless communication. Alternatively, the detector 806 may communicate with the sound module by infrared or laser means. The RF channel selector is suitable for enabling use of multiple systems in close proximity without interference. For example, a single home may use more than one system 800 and can adjust each one of the systems to use a different RF channel, thereby eliminating RF interference among the systems. The detector 806 operates such that it emits a pulse signal on activation rather than a continuous signal. A pulse signal decreases the chance of RF interference and reduces consumption of battery power under normal use.

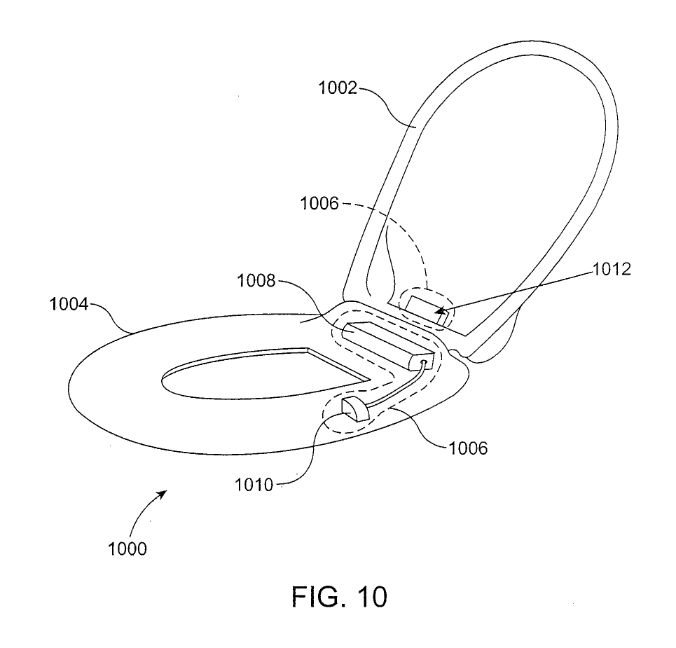

FIG. 10 illustrates a toilet seat/lid combination 1000 for a sound privacy system constructed in accordance with the present invention. In the system 1000 illustrated in FIG. 10, a toilet lid 1002 is hinged to a toilet seat 1004, and a detector 1006 (indicated by the dashed lines) is fixed to the toilet lid and seat. The detector 1006 includes a control module 1008 fixed to the underside of the seat 1004 and a sensor module that includes a pressure sensor 1010 and a motion sensor 1012. The sensor module can include both a pressure sensor and a motion sensor, or just one or the other. If both types of sensor are included, then a control module sensor selector (FIG. 7) determines which sensor has been activated (that is, which sensor is generating a signal) and determines which sensor to use and in which combination, as noted above.

The detector 1006 senses motion of the object to which it is attached or fixed. If the motion sensor 1012 is attached to the toilet lid 1002, then the sensor detects when the lid is moved. That is, the motion sensor detects a change in position or inclination of the object to which it is attached. If the motion sensor 1012 is attached to the toilet seat 1004, then the sensor detects when the seat is moved. In addition, the motion sensor 1012 may be configured to detect motion in the vicinity of the object to which it is attached, such as with infrared or laser detection of persons moving in the vicinity of the toilet lid and seat.

As with the previously described embodiments, the detector 1006 is in direct wireless communication with the sound system 100 (FIG. 4) for controlling activation of predetermined masking sounds, such as music, when one of the sensors 1010, 1012 is triggered or activated in accordance with the configured control module operation. It should be understood that the detector 1006 controls operation of an associated sound module, such as the sound module 100 having the construction illustrated in FIG. 4. Therefore, the detector 1006 includes an RF channel selector, a transmitter, and an antenna as illustrated above for the FIG. 7 block diagram, so the detector can communicate with the associated sound module via wireless communication. Alternatively, the detector 1006 may communicate with the sound module by infrared or laser means.

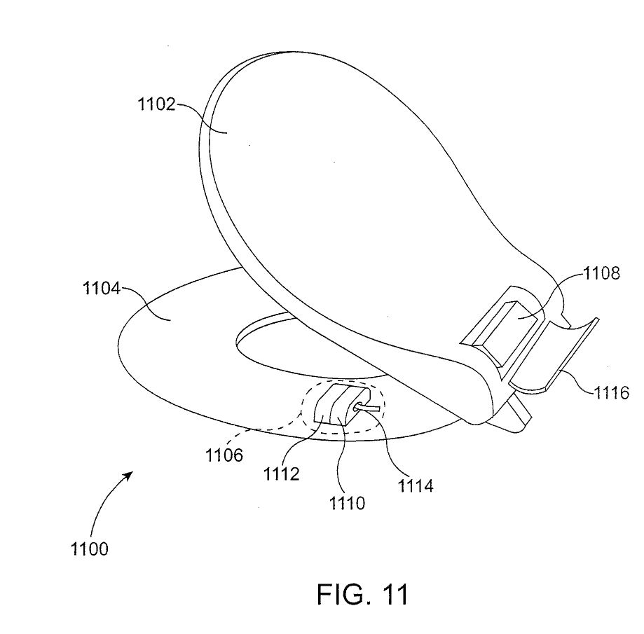

FIG. 11 illustrates another embodiment of a toilet seat/lid combination 1100 for a sound privacy system constructed in accordance with the present invention. In the system 1100, a toilet lid 1102 is hinged to a toilet seat 1104, and a detector 1106 that includes sensors (indicated by the dashed lines) fixed to the toilet seat as well as a control module 1108 fixed to the lid 1102. The detector 1106 includes a pressure sensor 1110 and a motion sensor 1112. A wire 1114 is shown connecting the sensors to the control module for communicating the sensor signals to the control module. The signals can alternatively be communicated to the control module 1108 using wireless means. In this way, the control module operates as a receiver for the signals from the sensors 1110, 1112.

In the FIG. 11 system 1100, the control module 1108 of the detector is located within the toilet lid 1102 and is accessed via a lid or access panel 1116 in the toilet lid. Access to the control module is necessary for setting the control module operation and for routine maintenance such as battery replacement. The control module can optionally be set for operation in accordance with user preferences, so as to generate sounds in accordance with such preferences. For example, if it is desired for users to leave the toilet lid down upon leaving the toilet, the system can be set via user controls so that a recorded message can be generated if the motion sensors detect that the lid remains in a raised position after the pressure sensor indicates that no weight is on the toilet seat. In addition, the system may be configured so that it does not generate sound, regardless of the lid position, if the pressure sensor indicates that no weight has been placed on the toilet seat. Such options can be set automatically in the control module in firmware or the like, or such options can be selectable by the user via a control keypad on the control module or by some other arrangement for a user to indicate a preferred operational configuration. Alternatively, the system can be configured so that such options are set and controlled via the sound system. That is, the sound system can be provided with a user keypad or other input receiving means to accept operational parameters and settings from a user, such that the detector and control module will operate in accordance with the desired functionality such as described above.

As with the previously described embodiments, the detector 1106 is in direct wireless communication with the sound system 100 (FIG. 4) for controlling activation of predetermined masking sounds, such as music, when one of the sensors is triggered or activated in accordance with the configured control module operation. It should be understood that the detector 1106 controls operation of an associated sound module, such as the sound module 100 having the construction illustrated in FIG. 4. Therefore, the detector 1106 includes an RF channel selector, a transmitter, and an antenna as illustrated above for the FIG. 7 block diagram, so the detector can communicate with the associated sound module via wireless communication. Alternatively, the detector 1106 may communicate with the sound module by infrared or laser means.

The foregoing description is considered as illustrative only of the principles of the invention. Further, since numerous modifications and changes will readily occur to those skilled in the art, it is not desired to limit the invention to the exact construction and process shown as described above. Accordingly, all suitable modifications and equivalents may be resorted to falling within the scope of the invention as defined by the claims that follow. The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

The present invention has been described above in terms of presently preferred embodiments so that an understanding of the present invention can be conveyed. There are, however, many configurations for sound privacy systems not specifically described herein but with which the present invention is applicable. The present invention should therefore not be seen as limited to the particular embodiments described herein, but rather, it should be understood that the present invention has wide applicability with respect to sound privacy systems generally. All modifications, variations, or equivalent arrangements and implementations that are within the scope of the attached claims should therefore be considered within the scope of the invention.

I claim:

1. An apparatus for providing masking sounds in the vicinity of a toilet, the apparatus comprising:
a detector positioned in the vicinity of a toilet, wherein said detector comprises a transmitter; and
a receiver that is in direct wireless communication with said detector and thereby activates a sound system, wherein said sound system is capable of emitting masking sounds.

2. The apparatus of claim 1, wherein said detector is positioned on the lid of a toilet and when the toilet lid is in a horizontal position said detector communicates to said receiver a signal that turns said sound system off and when the lid is in a vertical position the detector communicates a signal to said receiver that turns said sound system on.

3. The apparatus of claim 1, wherein said receiver is located in a switchable power outlet into which said sound system is plugged.

4. The apparatus of claim 1, wherein said receiver is located in said sound system.

5. The apparatus of claim 1, wherein said detector further comprises a tilt switch.

6. The apparatus of claim 5, wherein said tilt switch is a ball bearing switch.

7. The apparatus of claim 1, wherein said detector further comprises an emitter that emits a beam, wherein said beam forms a sensing zone; a receiver that detects said beam when it is reflected from an object wherein said transmitter communicates directly with a receiver positioned within said sound system.

8. The apparatus of claim 1, wherein said receiver includes a supplementary device.

9. The apparatus of claim 8, wherein the supplementary device comprises an air cleaner.

10. A method for masking the sound of eliminating, the method comprising:
positioning a detector in the vicinity of a toilet;
sensing use of said toilet; and
activating a wireless sound system set at a predetermined volume to mask sounds of eliminating.

11. The method of claim 10, wherein said detector is positioned on the toilet lid.

12. The method of claim 11, wherein said detector comprises a tilt switch.

13. The method of claim 12, wherein said tilt switch is a ball contact switch.

14. The method of claim 11, wherein the detector comprises at least one ball contact switch that detects horizontal movement and at least one ball contact switch that detects vertical movement.

15. The method of claim 10, wherein said detector comprises an emitter that emits a beam, wherein said beam forms a sensing zone; a receiver that detects said beam when it is reflected from an object wherein said transmitter communicates directly with a receiver positioned within said sound system.

16. The method of claim 10, wherein activating further includes activating a supplementary device.

17. The method of claim 16, wherein activating a supplementary device comprises activating an air cleaner.

18. A method of turning on a sound system, the method comprising:
attaching a motion detector to the lid of the toilet whereby the motion detector detects motion of the lid between horizontal and vertical and the motion detector controls activation of the sound system such that the sound system is switched off in the horizontal position of the lid and the sound system is switched on in the vertical position of the lid;
raising the lid to a vertical position to switch the sound system on; and
lowering the lid to a horizontal position to switch the sound system off.

19. A sound masking system comprising:
a sound module that generates masking sounds;
a detector that detects presence of a person in the vicinity of a toilet and in response activates the sound module so it generates the masking sounds such that the generated masking sounds are sufficient to mask noises of bodily functions in the vicinity of the toilet.

20. The sound masking system as defined in claim 19, wherein the detector comprises:
a control module;
at least one motion sensor fixed to either a lid of the toilet or a seat of the toilet such that the motion sensor detects motion in the vicinity of the toilet.

21. The sound masking system as defined in claim 20, wherein the detector includes a motion sensor fixed to the toilet lid and a motion sensor fixed to the toilet seat.

22. The sound masking system as defined in claim 20, wherein the detector further includes a pressure sensor that detects when a weight is placed on the toilet seat.

23. The sound masking system as defined in claim 20, wherein the system includes at least two sensors form a set comprising motion sensors and presence sensors, and the control module is configured such that the sound module is activated if a first sensor of the set is activated followed by a second sensor of the set being activated, and the sound module is not activated otherwise.

24. The sound masking system as defined in claim 19, further including a user keypad for receiving operational configuration settings for operation of the system.

25. The sound masking system as defined in claim 24, wherein the user keypad is located at the sound module.

26. The sound masking system as defined in claim 24, wherein the user keypad is located at the detector.
